(19) United States
(12) Patent Application Publication
(10) Pub. No.: US 2023/0174927 A1
(43) Pub. Date: Jun. 8, 2023

(54) LACTOBACILLUS SAKEI FOR ALLEVIATING ALLERGY AND PREPARATION METHOD THEREOF

(71) Applicants: Zhejiang Gongshang University, Hangzhou (CN); ZHEJIANG LIZIYUAN FOODSTUFF CO., LTD, Jinhua City (CN)

(72) Inventors: Linglin Fu, Hangzhou (CN); Shunyu Wang, Hangzhou (CN); Chong Wang, Hangzhou (CN); Qiaozhi Zhang, Hangzhou (CN); Jinru Zhou, Hangzhou (CN); Huan Li, Hangzhou (CN); Gang Yu, Hangzhou (CN)

(73) Assignees: Zhejiang Gongshang University, Hangzhou, ZJ (CN); ZHEJIANG LIZIYUAN FOODSTUFF CO., LTD, Jinhua City, ZJ (CN)

(21) Appl. No.: 17/985,500

(22) Filed: Nov. 11, 2022

(30) Foreign Application Priority Data
Dec. 6, 2021 (CN) 202111475886.1

(51) Int. Cl. C12N 1/20; A61K 35/747; A61P 37/08; A23L 33/135

(52) U.S. Cl. CPC C12N 1/205; A61K 35/747; A61P 37/08; A23L 33/135; C12R 2001/225

(57) ABSTRACT

The present invention discloses Lactobacillus sakei for alleviating allergy and a preparation method thereof, and particularly Lactobacillus sakei for alleviating wheat-dependent, exercise-induced anaphylaxis (WDEIA). The Lactobacillus sakei is classified and named as Lactobacillus sakei. The Lactobacillus sakei has been preserved in China Center for Type Culture Collection since Apr. 12, 2003, with a preservation number of CCTCC DB 20082413. The Lactobacillus sakei can regulate the immune function of a human body to prevent/treat WDEIA by reducing the contents of specific IgE and histamine in serum of WDEIA patients.

### TECHNICAL FIELD

The present invention relates to the technical field of microorganism, and in particular to Lactobacillus sakei for alleviating wheat-dependent, exercise-induced anaphylaxis, as well as a preparation method, a microecological preparation and an application thereof.

### BACKGROUND

Gluten-related diseases belong to an emerging worldwide disease, with a global prevalence of approximately 5% in general population. Gluten-related diseases include celiac disease, non-celiac disease, gluten allergy and wheat allergy. When a specific allergic reaction is involved, namely wheat-dependent, exercise-induced anaphylaxis (WDEIA), which specifically occurs in physical exercises after wheat ingestion. It is clinically characterized by an allergic reaction within 1-4 hours after wheat ingestion and physical exercise, usually with symptoms ranging from local or generalized urticaria to respiratory, gastrointestinal, or cardiovascular disease, including angioedema, respiratory difficulty, collapse and shock. The intake of aspirin and alcohol, the presence of infection in a body, stress or female hormone stimulation can replace or synergize exercise to cause hypersensitivity after wheat ingestion.

At present, the common measures to alleviate and control such WDEIA are to avoid the intake of food containing wheat products, including various cooked wheaten foods, biscuits and breads, or not to do strenuous exercise within 6 hours after eating wheat products. Gluten-removed wheat products also exist today, but these products lose some of nutrients, have an impact on production and processing, and cannot fundamentally alleviate or control the WDEIA.

### SUMMARY OF THE INVENTION

According to different probiotic strains, including Lactobacillus acidophilus, Lactobacillus rhamnosus, Lactobacillus casei, Lactobacillus paracasei, Lactobacillus reuteri and Lactobacillus plantarum, wheat allergy mouse models are established by means of mixed gastric infusion with sensitinogen wheat protein and an immunoadjuvant CT, probiotic strains that are beneficial for alleviating a food allergy (especially for alleviating WDEIA) are screened, and finally Lactobacillus sakei for alleviating WDEIA is screened out and obtained. Therefore, the present invention is intended to provide Lactobacillus sakei for alleviating WDEIA.

In order to achieve the above-mentioned objective, the present invention provides the following technical solution: Lactobacillus sakei for alleviating WDEIA, wherein the Lactobacillus sakei is classified and named as Lactobacillus sakei; the Lactobacillus sakei has been preserved in China Center for Type Culture Collection since Apr. 12, 2003, with a preservation number of CCTCC DB 20082413.

Preferably, the Lactobacillus sakei can reduce the contents of specific IgE and histamine in serum of WDEIA patients.

Preferably, the Lactobacillus sakei can be prepared into microcapsules, tablets or lyophilized powder by supplementing food excipients or medicinal carriers.

A preparation method of Lactobacillus sakei includes the above-mentioned Lactobacillus sake, also includes the following steps: treating and culturing traditional fermented milk samples, isolating and purifying strains from colonies of different characteristics according to colony characteristics, and preliminarily identifying lactic acid bacteria by Gram staining and a peroxidase experiment; performing colony morphology and microscope observation on the isolated strains to determine a target strain; activating the target strain with an MRS liquid medium, extracting DNA with a bacterial genomic DNA extraction kit, and amplifying16srRNA; and performing sequencing analysis after examining PCR products by 1.0% agarose gel electrophoresis.

The present invention has the following beneficial effects: the Lactobacillus sakei of the present invention can regulate the immune function of a human body to prevent/treat WDEIA by reducing the contents of specific IgE and histamine in serum of WDEIA patients.

### BRIEF DESCRIPTION OF THE DRAWINGS

FIG. 1 is the structural image of the Lactobacillus sakei of the present invention under an optical microscope.

FIG. 2 shows an effect of the Lactobacillus sakei of the present invention on specific IgE in serum of WDEIA patients.

FIG. 3 shows an effect of the Lactobacillus sakei of the present invention on the histamine content in serum of WDEIA patients.

### DETAILED DESCRIPTION OF THE INVENTION

The specific implementation modes of the present invention will be further described below in combination with the drawings, but it should be understood that the protection scope of the present invention is not limited by these implementation modes. It should be noted that unless otherwise noted, all the experimental methods used in the following embodiments are conventional methods; all the materials and reagents used in the following embodiments are commercially available.

For conditions not noted in the embodiments, conventional conditions or the conditions suggested by the manufacturer apply. The reagents or instruments not noted with manufacturer are all commercially available conventional products. In order to make the objectives, technical solutions and advantages of the present invention more clearly, the technical solutions in the embodiments of the present invention will be clearly and completely described below.

### EXAMPLE 1

The present invention provides Lactobacillus sakei for alleviating WDEIA, wherein the Lactobacillus sakei has been preserved in China Culture for Type Culture Collection (“CCTCC”) since Apr. 12, 2003, with a preservation number of CCTCC DB 20082413.

The Lactobacillus sakei is classified and named as Lactobacillus sakei, and the preservation site is Wuhan University in Wuhan, China.

In the example, the Lactobacillus sakei was round-ended straight bacterium, usually 0.9-1 μm, existing in the form of individual, in-pair or short chain. The Lactobacillus sakei was usually free of flagella but capable of moving. The Lactobacillus sakei was anaerobic, and the surface colony was about micron wide, convex, round, smooth, compact, white, and occasionally light yellow or deep yellow. The grower in the medium was even quite thick.

Further, the Lactobacillus sakei was prepared into edible microcapsules, tablets or lyophilized powder, containing a bacterial count of 1×(10⁸-10¹⁰) cfu/kg, and applied in preparing drugs, health care products or foods; the intake of the Lactobacillus sakei was 1×10⁸ to 1×10¹⁰ cfu per day.

Studies have found that the Lactobacillus sakei can regulate the organic immune function to prevent/treat WDEIA by reducing the contents of specific IgE and histamine in serum of WDEIA patients.

A main allergen of WDEIA is ω-5 prolamin (Tria 19). By using single karyocytes in serum and peripheral blood of WDEIA patients that the WDEIA patients have prolamin specific IgI antibody, and it has been identified that the ω-5 prolamin (Tria 19) is the main allergen. Blood redistribution during exercise leads to ischemia of the gastrointestinal mucosa, and undigested wheat proteins (mainly prolamin) enter the blood through the digestive tract, bind to specific IgE, and activate basophils and mast cells to degranulate and release inflammatory mediators, thereby resulting in the allergy. In WDEIA patients, basophils release high levels of histamine, the ω-5 prolamin specific IgA antibody levels are significantly elevated, and IL-10 is reduced. The Lactobacillus sakei of the present invention can inhibit the differentiation of Th0 to Th2, significantly regulate the elevated IgE and IL-4, enhance the differentiation of Treg and the secretion of IL-10 in mesenteric lymph nodes, and reduce the secretion of histamine by basophils to reduce the WDEIA, thereby providing a feasible approach for the prevention and treatment of WDEIA.

### EXAMPLE 2

The example provides a preparation method for the Lactobacillus sakei for alleviating allergy proposed in the above example, including the isolation and identification of strains, and the specific steps are as follows:

Isolation of Strains

A traditional fermented milk sample was properly diluted with sterile physiological saline, 100 μL of the diluted sample was smeared on an MRS (10 g of peptone, 5 g of yeast extract, 2 g of K₂HPO₄ (including 2.62 g of crystal water), 2 g of triamine citrate, 20 g of glucose, 0.05 g of MnSO₄ (0.198 g of MnSO₄.H₂O), 0.28 g of MgSO₄, 8 g of meat extract, 15-20 g of agar, 5 g of NaAc (anhydrous sodium acetate), 1 mL or 1 g of Tween-80, 1 L of distilled water, Ph 7.2) agar medium plate and cultured at 37 degrees Celcius for 48 h, then counting was performed, strains were isolated and purified from colonies of different characteristics according to the characteristics of the colonies, and lactic acid bacteria were preliminarily identified by Gram staining and peroxidase experiment.

The isolated strains were subjected to colony morphology and microscopic observation, and the G+ and catalase-negative bacilli were selected and further identified according to the method of Harrigan & McCance. The conformation of lactic acid was determined using D-lactate and L-lactate dehydrogenase kits. The carbohydrate fermentation modes of the lactic acid bacteria strains were determined using an API kit. The physiological and biochemical characteristics and phenotypic identification of the target Lactobacillus sakei were as shown in Table 1 below.

TABLE 1
Physiological and Biochemical Characteristics and Phenotypic
Identification of the Target Lactobacillus Sakei

| | Ammonia | Lactic | | |
|---|---|---|---|---|
| | Production | Acid | | |
| Cell | from | Con- | Fermentation | Result of API |
| Morphology | Arginine | figuration | Mode | Identification |
| Bacillus | — | DL | Homolactic | Lactobacillus |
| | | | Fermentation | Sakei |

Note:
In the results, “+” represents positive, and “−” represents negative.

The target strain was selected for activation with an MRS liquid medium, and the liquid culture condition was 37 degrees Celcius for 12 h-16 h. DNA extraction was performed using a bacterial genomic DNA extraction kit, followed by 16srRNA amplification. Universal primers were used for the 16srRNA amplification, wherein an upstream primer was pA (5′-AGA GTT TGA TCC TGG CTC AG-3′) and a downstream primer was pH (5′ GGC TAC CTT GTT ACG ACT 3′); a PCR system was 50 μL of The PCR reaction system included buffer solution containing MgCl₂ 1.5 mmol/L, dNTP 0.2 mmol/L, each of the primers 0.5 μmol/L, DNA template 1 μg/mL and 1U of Taq DNA polymerase. PCR conditions were: 95° C., 4 min; 35×[94° C. for 45 s, 55° C. for 45 s, 72° C. for 1 min]; 72° C., 5 min.

Sequencing analysis was performed after PCR products were examined by 1.0% agarose gel electrophoresis.

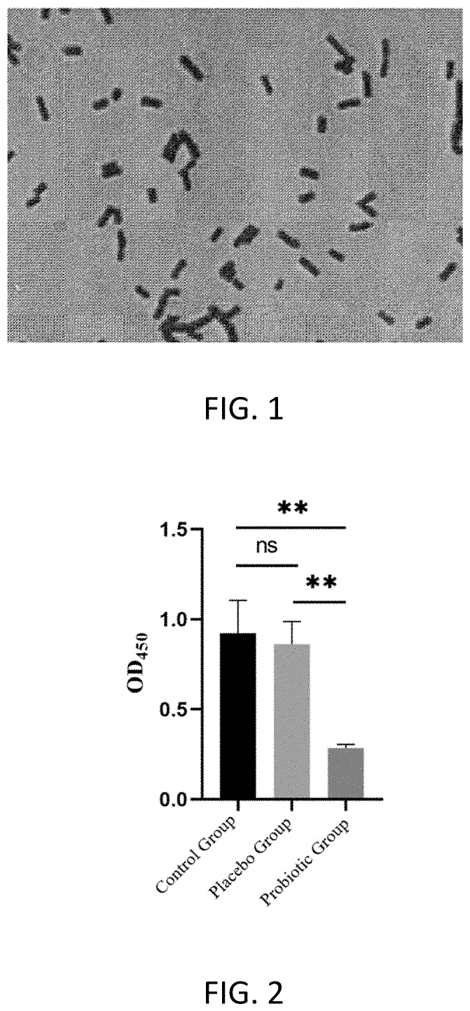

The Gram-stained Lactobacillus sakei was observed under an optical microscope, and the results showed that the Lactobacillus sakei was rod-shaped and had uniform morphology. The specific image under the optical microscope was as shown in FIG. 1.

### EXAMPLE 3

### Alleviation of WDEIA by Lactobacillus Sakei

10 WDEIA patients as research objects were randomly divided into a control group (n=3), a placebo group (n=3) and a probiotic group (n=4). During the test, the subjects in the probiotic group took separately packaged Lactobacillus sakei (2 g, with a viable count of 0.5×10¹⁰ CFU/g) within 1 h after lunch every day, and the subjects in the placebo group took placebo powder (2 g of dried porous dextrin) within 1 h after lunch every day, for 28 consecutive days. The food taken by all the subjects remained the same before and during the test. On Day 28 after taking the probiotic or placebo, blood samples were collected from the subjects, and high-speed centrifugation was performed to obtain serum for immune index detection.

Determination of specific IgE in serum: enzyme-linked immunosorbent assay (ELISA) was used for determination. Wheat prolamin was diluted with a PBS solution to 15 μg/mL, then fixed in a 96-well plate overnight at 4° C., washed with 200 μL of the PBS solution for 3 times, and blocked with 200 μL of 5% BSA at 25° C. for 2 h. Afterwards, the plate was washed for 3 times and incubated with 100 μL of serum (diluted by 1:30) of the 10 subjects at 37° C. for 1.5 h, and non-allergic serum was used as a negative control. After washing for 3 times, 100 μL of HRP-conjugated goat anti-human IgE (1:5,000) was added to each well for incubation at 37° C. for 1.5 h. Then 100 μL of a tetramethylbenzidine solution was added to each well for incubation at 37° C. for 15 min. The chromogenic reaction was terminated by 50 μL of 2 M H₂SO₄ solution. The optical density of each well was measured at 450 nm using a microplate reader (Molecular Devices, USA).

The results were as shown in FIG. 2 that compared with the control group, there was no significant change in the serum IgE level of the WDEIA patients in the placebo group; the serum IgE level of the WDEIA patients in the probiotic group after the Lactobacillus sakei intervention for 28 d was significantly lower than those in the control group and the placebo group. This indicates that Lactobacillus sakei intervention has a regulatory effect on WDEIA patients and can alleviate WDEIA

### EXAMPLE 4

### Alleviation of WDEIA by Lactobacillus Sakei

10 WDEIA patients as research objects were randomly divided into a control group (n=3), a placebo group (n=3) and a probiotic group (n=4). During the test, the subjects in the probiotic group took separately packaged Lactobacillus sakei (4 g, with a viable count of 1×10¹⁰ CFU/g) within 1 h after lunch every day, and the subjects in the placebo group took placebo powder (2 g of dried porous dextrin) within 1 h after lunch every day, for 28 consecutive days. The food taken by all the subjects remained the same before and during the test. On Day 28 after taking the probiotic or placebo, blood samples were collected from the subjects, and high-speed centrifugation was performed to obtain serum for immune index detection.

Determination of histamine in serum: the histamine in serum was determined using a human histamine ELISA kit (such as an ELISA kit sold by Thermo Fisher Scientific of Waltham, Mass. USA) according to the instructions, and the absorbance OD value of each well was measured at 450 nm.

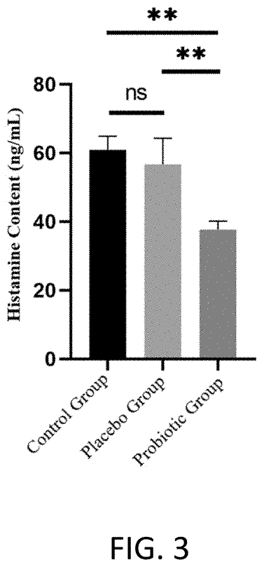

The results were as shown in FIG. 3 that compared with the control group, there was no significant change in the histamine level in serum of the WDEIA patients in the placebo group; the histamine level in serum of the WDEIA patients in the probiotic group after the Lactobacillus sakei intervention for 28 d was significantly lower than those in the control group and placebo group. This indicates that Lactobacillus sakei intervention has a regulatory effect on WDEIA patients and can alleviate WDEIA

In summary, the Lactobacillus sakei of the present invention can reduce the contents of allergy specific IgE and histamine in serum of WDEIA patients, thereby alleviating WDEIA. Therefore, the Lactobacillus sakei can be used to alleviate WDEIA.

It should be understood that the present invention is described by the implementation modes, and the embodiments are given only to clearly and completely explain the technical solutions proposed by the claims of the present invention, that is, to explain the claims. Therefore, when judging whether the technical solutions recorded in the specification are fully disclosed, full consideration should be given to the core ideas of the solutions confined by the claims. However, there are certainly other unrelated technical solutions in the specification that are proposed to solve the core technical problems, and the corresponding technical features and technical solutions belong to unnecessary technical features, rather than the main idea of the present embodiment. Thus, they can refer to inherent disclosure. Those skilled in the art can completely achieve the present invention in combination with the prior art and common general knowledge, so that it is unnecessary to give detailed description at all.

It should be stated that the above-mentioned embodiments are only used for explaining, rather than limiting, the technical solutions of the present invention. Although the present invention is explained in detail by reference to the preferred embodiments, those of ordinary skill in the art should understand that modifications or equivalent substitutions may be made to the technical solutions of the present invention without deviating from the spirit and scope of the technical solutions of the present invention, and all the modifications or equivalent substitutions should fall into the scope of the claims of the present invention.

What is claimed:

1. A microorganism for alleviating wheat-dependent, exercise-induced anaphylaxis (WDEIA) including,
a Lactobacillus sakei that has been preserved in a China Center for Type Culture Collection since Apr. 12, 2003, with a preservation number of CCTCC DB 20082413,
wherein
the Lactobacillus sakei is classified and named as Lactobacillus sakei;
the Lactobacillus sakei is used to alleviate WDEIA.

2. The Lactobacillus sakei for alleviating WDEIA of claim 1, wherein the Lactobacillus sakei reduces the contents of specific IgE and histamine in serum of WDEIA patients.

3. The Lactobacillus sakei for alleviating WDEIA of claim 1, wherein the Lactobacillus sakei is prepared into microcapsules, tablets or lyophilized powder by supplementing food excipients or medicinal carriers.

4. A method for preparing a Lactobacillus sakei, comprising the following steps:
treating and culturing traditional fermented milk samples;
isolating and purifying strains from colonies of at least one unique characteristic according to a plurality of colony characteristics;
preliminarily identifying lactic acid bacteria by Gram staining and a peroxidase experiment;
performing colony morphology and microscope observation on the isolated and purified strains to determine a target strain;
activating the target strain with an MRS liquid medium;
extracting DNA with a bacterial genomic DNA extraction kit, and amplifying16srRNA; and
performing sequencing analysis after examining PCR products by 1.0% agarose gel electrophoresis.

5. The method of claim 4, wherein the Lactobacillus sakei can reduce the contents of specific IgE and histamine in serum of WDEIA patients.

6. The method of claim 4, including the step of preparing the Lactobacillus sakei into microcapsules, tablets or lyophilized powder by supplementing food excipients or medicinal carriers.
